(19) United States
(12) Patent Application Publication
(10) Pub. No.: US 2015/0045784 A1
(43) Pub. Date: Feb. 12, 2015

(54) IMPLANT DEVICE WITH SPINE AND C-RING AND METHOD OF MAKING, DELIVERING, AND USING SAME

(71) Applicant: Christopher Gerard Kunis, Escondido, CA (US)

(72) Inventor: Christopher Gerard Kunis, Escondido, CA (US)

(21) Appl. No.: 14/316,084

(22) Filed: Jun. 26, 2014

Related U.S. Application Data
(60) Provisional application No. 61/957,257, filed on Jun. 26, 2013.
(60) Provisional application No. 61/957,309, filed on Jun. 27, 2013.
(60) Provisional application No. 61/878,340, filed on Sep. 16, 2013.
(60) Provisional application No. 61/957,371, filed on Jul. 1, 2013.
(60) Provisional application No. 61/902,498, filed on Nov. 11, 2013.

(51) Int. Cl. A61B 18/08; A61B 17/00; A61B 18/10

(52) U.S. Cl. CPC A61B 18/082; A61B 18/10; A61B 17/00234; A61B 2018/00416
USPC 606/30; 29/458; 29/428; 29/825

(57) ABSTRACT

An implant device comprising at least one spine and at least one c-ring is provided, and may optionally include a stabilizing element and/or tissue penetrating features. A method of treating a malady with such implant device may also be provided, as well as a method of delivering and/or removing the implant device. A delivery system may be included, as well as a kit that includes the delivery system and implant device.

### CROSS REFERENCE TO RELATED APPLICATIONS

This application claims benefit under 35 USC 119(e) of U.S. Provisional Patent application 61/957,257, filed Jun. 26, 2013, entitled “Device and Method for Treatment of Mammilian Arrhythmias, Neurological Disorders and Other Maladies;” U.S. Provisional Patent application 61/957,309, filed Jun. 27, 2013, entitled “Device and Method for Treatment of Left Atrial Apendage Ligation in Mammilian and Other Maladies;” U.S. Provisional Patent application 61/878,340, filed Sep. 16, 2013, entitled “Device and Method for Treatment of Mammilian Arrhythmias and Neurological Disorders and Other Maladies;” U.S. Provisional Patent application 61/957,371, filed Jul. 1, 2013, entitled “Device and Methods for Implant with Spine and C-ring for Treating a Series of Maladies Within Mammilian Body;” and U.S. Provisional Patent application 61/902,498, filed Nov. 11, 2013, entitled “Implant Device with Spine and C-ring and Method of Making, Delivering, and Using Same”, each of which is incorporated by reference herein in their entirety.

This application may also be related to commonly owned U.S. patent application Ser. No. 14/312,118, entitled “Implant Device With Stabilizer,” filed Jun. 23, 2014, the contents of which are incorporated herein in its entirety by references.

### FIELD OF INTEREST

This application relates to implants useful in treating a series of maladies within a mammalian body, and particularly to implant devices useful in blood vessels and organs.

### BACKGROUND

Atrial fibrillation (AF) is a common and dangerous disease. It is the most common arrhythmia, and accounts for approximately ⅓ of all hospitalizations due to heart rhythm disorders. In addition, atrial fibrillation ablation patients have a greatly increased risk of pulmonary vein stenosis as a result of energy based therapies, in addition there is a 6× increase in stroke mortality.

Current first-line therapies for atrial fibrillation include the use of anti-arrhythmic drugs and anti-coagulation agents. Drugs are useful at reducing symptoms, but often include undesirable side effects. Anti-coagulation agents can reduce the risk of stroke, but often increase the risk of bleeding.

Second-line therapies include surgical and catheter ablation. However, the same are associated with high complication rates, such as pulmonary vein (PV) stenosis, long procedure times, and limited clinical evidence. In addition, their administration typically requires extensive training in the use and installation of complex ablation technology.

Pulmonary vein (PV) isolation is the cornerstone of ablation strategies and these procedures are growing annually. Therefore, the number of complications, such as PV stenosis, is also increasing. Additionally, there is a greater need today, more than ever, for a device for the treatment of these complications, as well as idiopathic pulmonary fibrosis.

Traditional stents have also been used to treat stenosis; such stents have a tubular, mechanically constrained structure. The tubular structure helps with stability during and after placement in a blood vessel however, this implant design has limitations and significant clinical challenges. Furthermore, the tubular structures documented in the prior art are traditionally formed by use of various methods.

Such tubular and constrained implant structures suffer from a critical issue when it comes to conformation to vascular tissue/structures of a mammalian body, wherein conformation to a vessel wall that is not perfectly round is a challenge for a tubular constrained stent. As a matter of fact, most vascular structures are more oval or oblong in nature than round or circular. A tubular constrained structure, such as a typical stent, is inherently stable after being deployed within a vessel, however, the traditional tubular implant will not expand in a radial or longitudinal direction or both with an equal distribution of force to surrounding vessel tissue needed to achieve the optimal clinical outcome for a given patient. More specifically, the larger vessels in the mammalian body would greatly benefit from a freeform, stable implant that is capable of generating a substantially equal force radially or longitudinally along an axis of a vessel.

### SUMMARY

The present inventive concepts were conceived based on knowledge and understanding of optimal implant design and the shortfalls of current implant designs and functionality.

The implant devices and methods described herein may be especially suited for treating Arrhythmias, Hypertension, stenosed cardiovascular vessels, Abdominal Aortic Aneurysms (AAA), or other maladies. An implant device in accordance with such concepts employs the beneficial features of a tubular constrained structure with the added benefits of the free-form features employed to enable an equal distribution of force along the axis of the vessel within a mammalian body. However, historically a free-form implant design has posed many engineering challenges, both clinically and functionally, and a delivery system for such a free-form implant design can be challenging to engineer to achieve the high level of repeatability and reliability needed in design and development of medical devices.

An implant device in accordance with aspects of the present invention includes the stability characteristics of a traditional tubular stent implant with the features of a free-form implant design that enables a substantially equal distribution of load against surrounding tissue, e.g., along the axis of said vessel and implant device. Therefore, the present implant device is structured and functions differently from those currently available devices for the treatment of AAA diseased patients, as an example.

In various embodiment, such an implant device may be made to have a biologically inert covering or coating, such as ePTFE or Dacron.

In accordance with aspects of the invention, a superstructure of the implant device includes a spine supporting a c-ring or series of c-rings. The spine can be straight, substantially straight, or curved, e.g., to improve the flexibility of said superstructure. The implant device can include a single or series of otherwise unconnected or unconstrained c-rings, which can have ends that extend past each other to form a slip-ring. The c-ring (or slip-ring) design may enable the ends of the c-ring to pass by each other, for example when the implant device is contracted or expanded. As an example, when viewed from an end, a c-ring can have a circular or circle-like shape, or can have a different geometric shape, e.g., an oval, an octagon, a hexagon, a pentagon, or other such shapes. In some embodiments, ends of a c-ring may begin and end in the same plane, e.g., similar to meshing of teeth or abutting ends.

The implant device geometric shape may be designed to improve conformability to the vessel or other structures within a mammalian body following implantation. By changing the geometry of the implant, the coil and appendage may be mutually conformed, and the inward radial force equalized along the circumference of the inner surface of the tissue. The implant may have the above-noted shapes at the proximal end, but may employ a different shape at the distal end of the implant such as a funnel-like shape to conform to an ostium or antrum of a vessel a such as with a pulmonary vein.

An implant device in accordance with the present invention can, therefore, include one or more c-rings affixed to or integral with at least one spine, which enables the implant device to conform to various volumes within a mammalian body (e.g., blood vessel). The implant device can have a self-adjusting design, e.g., the ends of the c-ring formed in the shape of a ring with unconnected ends that can be made to pass each other for expansion and contraction, while exhorting an outward radial force against the surrounding tissue when expanded. Since the ends of the unconstrained c-ring or c-rings extend past each other, a self-adjusting c-ring is achieved that, even for an oversized vessel, enables the c-ring and implant device to conform to variations in tissue structures within a mammalian body.

Unlike currently available stent designs having been formed as a tubular closed and constrained implant, an implant device in accordance with the present invention improves the ability of the implant device to conform, as well as seal the implant device in place to reduce the chance for implant migration and, more importantly, provides a better mechanical design to seal blood flow from passing between the outside surface of the implant device and vessel wall, known in the industry as endoleaks—a complication well documented in the design and clinical use of currently available implants for the treatment of AAA.

Furthermore, the implant device can preferably employ a novel mechanical feature referred to herein as a Pressure Differential Feature (PDF). The PDF is shaped like a ventury or a blunt end of a wing shaped in a circular pattern and located within the implant device. This element of the implant device may be affixed to said implant prior to delivery within a body or may be an adjunctive element delivered and deployed after the covered implant-like structure is delivered, making the delivery a multi-part or multi-staged procedure. The PDF element is designed to lower the pressure around the area of an aneurysm, for example, and an implant device may include more than one PDF depending on the clinical need. This design feature is located in the blood flow and works on the same principal as a wing, such as with Bernoulli's Law.

By using this PDF mechanism within the implant device, fluid flow is increased in a localized area of a vessel or other location within a body, while reducing the forces at the aneurismal location. This method can also be used for neurological vessels and other locations within a body, in addition to uses for AAA. This design and method of use will be superior to current implant designs in that the inventive implant device can use common physics and fluid dynamics to route the forces away from the damaged vessel area, enabling the vessel to heal and improve the strength of the damaged area without the need for a covering or covered stent structure to absorb the load of the outward forces known to create the aneurismal site. The inventive implant device may employ one or more PDF's in the system to divert the pressure away from the aneurismal location of a vessel within a mammalian body.

Other aspects of the present implant device will enable the implant device to facilitate repair of surrounding tissue at the location of the aneurysm to biologically improve the vessel structure by forcing the body to proliferate endothiealal, fibrogen cellular matrix to the treatment site.

In accordance with another aspect of the invention, an implant device can be provided that includes at least one c-ring and at least one stabilizer element, which can mitigate or substantially eliminate tipping or other misalignment of a delivered implant device.

In some embodiment, the stabilizer element can take the form of at least one stabilizing wire, e.g., a non-straight wire. In some embodiments, the non-straight stabilizing wire can have a zigzag (or sinusoidal or square wave) shape. In other embodiments, the stabilizing wire can have another shape. For example, if there is a plurality of c-rings, one or more spines can be disposed between c-rings, and one or more c-rings can include a stabilizer element.

In some embodiments, the c-ring(s) can be oriented substantially crosswise in a blood vessel and the spine(s) (e.g., one or more spines) can be oriented substantially lengthwise, at the delivery site within the body, e.g., within a blood vessel.

The implant device can be delivered to a delivery site or target location using a delivery system, such as a catheter system used to deliver and/or implant other types of devices within a mammalian body. Therefore, such a delivery system design can have many uses and applicability for a wide range of clinical modalities for use within a mammalian body. As an example, in various embodiments, the implant device may be delivered using a delivery system that employs a balloon or other expandable device located at the distal end of a catheter and containment sheath designed to protect the implant device prior to and during delivery within a mammalian body.

The delivery system can, therefore, take the form of a catheter to be percutaneously introduced into a mammalian body to diagnose, treat, and/or potentially cure a number of clinical modalities that include, but are not limited to, Atrial Fibrillation, Hypertension, Left Atrial Appendage Closure, AAA and a number of other percutaneously treated maladies.

In another aspect of the invention, provided is a kit for treating a malady by deploying an implant device in a mammalian, including the above-noted implant device, and a delivery system. The delivery system can include a catheter and a distally mounted balloon or other expandable element. The expandable element could be made of a mesh like material that will enable the element to be radially dilated to conform to said tissue of a vessel or other such as a chamber in a body. The balloon element or expansion device enables blood to flow freely thru the vessel during deployment, for optimal implant use and delivery. The delivery device has a distal end, such that upon deployment of the implant device from the distal end, a longitudinal axis of the implant device is substantially collinear with a longitudinal axis of the vessel chamber or appendage.

The delivery catheter can have an inner shaft or guide wire lumen, and an external shaft, connected to one or more of the catheter shafts is a balloon or balloon like element that can be radially dilated with hydraulic or mechanical or both to inflate or radially dilate said balloon element. The shafts that form the delivery catheter may move independent from each other in both a longitudinal as well as radial motion. The expandable delivery element may also enable the implant to be crimped to an exterior surface of said delivery element for delivery and final positioning within a mammalian body. The implant crimped to said balloon may also enable a telescopic catheter configuration to aid in deployment and final positioning within a body. The crimped implant device along an axis of said balloon and said catheter shaft or shafts may also employ a protective tube that is incorporated into the outer or inner shaft and cover said implant, crimped to said delivery element connected to said catheter shaft forming the “delivery system”.

In another delivery system embodiment, a catheter having an inner shaft and outer shaft with an adjoining balloon located on said distal end of the catheter to which an implant maybe mounted externally to or internally to the balloon surface for delivery into a body and enabling a radial release mechanism by rotating said catheter releases said implant into a body, the balloon then is inflated or radially dilated to position and affix said implant within said body. Said implant having a smaller cross-sectional profile prior to and during delivery into said mammalian body then expanding or radially extended into a larger diameter then the implant state prior to delivery into the body. The balloon element may be expandable by gas, hydraulic, mechanical, electronic, or mechanical methods to increase the balloon diameter during deployment. The balloon element may be formed by means of a mesh-like material configured to substantially form a balloon structure or a polymer or metallic material. The balloon element may be affixed to said catheter shaft by adhesive, thermal connection or maybe formed by injection or blow molding or other molding or forming methods. Here, instead of a balloon, any expandable deliver element could be used, e.g., an expandable basket.

Implementations of the invention may include one or more of the following. The inserting may include delivering the implant device to the vessel through or exterior to a vessel, e.g. an aorta. The vessel may be a vein, aorta, or renal artery or any other vessel or chamber in a body. The inserting or deployment may include delivering the implant into the aorta or other similar structures. The method may further include administering local anesthesia and not general anesthesia to the patient. The inserting may further include pushing the implant device through the catheter with a pushing mechanism or means. The pushing mechanism for means may be coupled to the implant device using a grabbing means. The catheter or implant device may include electrical elements to enable mapping of cardiac signals prior to, during and after deployment of said implant occurs. This may include determining the sizes of an aorta, and may further include delivering at least one implant device to said aorta or a multitude of locations within a body or chambers.

The method may further include delivery and implantation of multiple implants with an optional stabilizer element such that these stabilizer elements interconnect like railroad cars to enable multiple devices in a single vessel, vein or chamber in the body. The inner connecting elements of said implant device may also enable multiple implants to be delivered in a single deployment into a multitude of vessels or chambers in a single catheter application. The malady may be AAA, an aorta, neurological disorders, hypertension, atrial fibrillation, stenosed arteries or a combination of Hypertension and Atrial fibrillation or other maladies.

The method may further include inductive heating process by means of electromagnetic fields or other inducing a local reaction with the implanted device enabling a local heating effect sufficient to aid in deployment of said implant, dilatation of said balloon element or both for deployment, in addition the heating method may also enable ablation and necroses tissue sufficiently to slow or block electrical signals known to trigger these diseases. The method may further include recapturing the implant device after the inserting by novel means of fixation to a freeform implantable device. The compression of the tissue in a mammalian body of said vessel, nerve or nerve bundles, Ion channel(s) in adjacent tissue sufficiently to block or retard any signals traveling along the axis of said vessels. This may include compressing the first one to five cellular layers of the adjacent tissue and neurological bundles. The mapping may be performed both before the inserting and after the inserting. The compression may be such that the delay is caused in conduction of at least 5%.

In another aspect, the invention is directed to a method for treating a malady, with the herein described implant device. Implementations of the invention may include one or more of the following. The method may further include selecting a radius of the c-ring of the implant device to be at least two times the radius of an aorta. The implant device may also include the use of micro circuits or electrical components, such as electrodes, diodes and capacitors etc. for monitoring vital characteristics of a mammalian or interpretation of information wirelessly transmitted from said implant within a body to an internal or external wireless device with software to aid in the interrogation of intracardiac electrograms or many other arrhythmia or neurological defects or diagnosis.

Implementations of the invention may include one or more of the following. The method may further include activating a circuit or plurality of electrodes on the delivery system, implant or both, the electrodes distributed along the implant or catheter outer shaft. The method may further include rotating the catheter at least partially during the activating, thereby causing deployment of said implant element into tissue. The method may further include inserting an implant by means of a delivery catheter device or placed directly during a surgical procedure into the vessel before, during or after said ablation method occurs, the implant device including a spine, c-ring or other such element with optional tissue penetrating elements, and optional stabilization element. The method may further include inserting an implant device into the vessel before or chamber, during or after said ablation method occurs, the implant device including a spine, c-ring and optional tissue penetration elements, and optional stabilization element.

In various embodiments, a method may further include a method of introduction into said mammalian body, navigation of said delivery catheter to a target location within a body and thereby release of said implant element within a mammalian body for treatment of a malady and procedure being performed under only local anesthesia rather than requiring general anesthesia.

In accordance with one aspect of the inventive concept, provided is an implant device, comprising at least one spine and at least two c-rings coupled together by the at least one spine.

In various embodiments, the at least two c-rings can be maintained in a substantially parallel orientation by the at least one spine.

In various embodiments, ends of at least one c-ring can overlap in a slip ring closure.

In various embodiments, ends of at least one c-ring can have a cut ring closure, wherein the ends do not overlap.

In various embodiments, ends of at least one c-ring can have mating teeth.

In various embodiments, one or more of the at least two c-rings can be configured to self-adjust to accommodate a shape of a target location within a vessel of a body.

In various embodiments, one or more of the c-rings can have a memory shape and can take a compressed state when constrained and automatically expands when such constraint is removed.

In various embodiments, the implant device can further comprise at least one stabilizer element coupled to at least one c-ring.

In various embodiments, the stabilizer element can be a wire laterally extending from the at least one c-ring.

In various embodiments, the stabilizer element can be a wire formed in the shape of a zigzag.

In various embodiments, the implant device can further comprise at least one tissue penetration element configured to increase frictional or mechanical resistance against inner walls of a vessel.

In various embodiments, the at least two c-rings can include an end c-ring having a funnel-like shape.

In various embodiments, the funnel-like shape can be configured to conform to an ostium or antrum of a vessel.

In various embodiments, the at least two c-rings can have fingers disposed around a common central axis.

In various embodiments, the at least one spine can include at least two helical ribbon spines that wrap at least partially around the common central axis.

In various embodiments, the at least two helical ribbon spines can bow out and away from the common central axis.

In various embodiments, the at least one spine can include a plurality of spines that couple together the at least two c-rings.

In various embodiments, two or more of the plurality of spines can couple together two c-rings.

In various embodiments, the implant device can further comprise a biologically inert coating.

In various embodiments, the implant device can further comprise a coating including one or more of drugs, biologics, chemicals, or combinations of one or more thereof.

In various embodiments, the implant device can further comprise a coating including a chemical ablation reagent.

In various embodiments, the at least one spine and the at least two c-rings can be formed from a single sheet of material.

In various embodiments, the implant device can be configured to deliver a force against tissue of the vessel in a range of between about 0.5 g/mm2 and about 300 g/mm2.

In various embodiments, the implant device can be configured to deliver a radial force against tissue of a mammalian vessel at a target location that is sufficient to cause necrosis or apoptosis in the tissue in a deployed state, the necrosis or apoptosis sufficient to or delay electrical, neurological signal conduction traveling along an axis of the vessel and/or within an adjacent chamber in mammalian.

In various embodiments, the implant device can be configured to deliver a radial force against tissue of a mammalian vessel at a target location that is sufficient to compress at least one Ion channel in the adjacent tissue sufficient to delay electrical or neurological signals traveling along an axis of the vessel and/or within an adjacent chamber in mammalian.

In various embodiments, the implant device can further comprise a micro-circuit configured to measure or monitor a value of electrical conduction propagating along the axis of a mammalian vessel within which the implant device is deployed.

In various embodiments, the implant device can further comprise one or more electrodes disposed in or on the at least one substrate and in communication with the micro-circuit, the one or more electrodes configured for sensing conditions within the vessel and/or delivering energy to the vessel.

In various embodiments, the one or more electrodes can include ablation electrodes, mapping electrodes, or ablation and mapping electrodes.

In various embodiments, the micro-circuit can be configured to use an Ionic exchange with the vessel to charge a battery of the micro-circuit.

In various embodiments, the micro-circuit can be configured to measure and/or monitor a value of electrical conduction propagating along the axis of the vessel.

In various embodiments, the micro-circuit can be further configured to wirelessly transmit an indication of the electrical conduction in mammalian.

In various embodiments, the micro-circuit can be configured to receive an electromagnetic signal and to inductively heat the vessel in response to the electromagnetic signal.

In accordance with other aspects of the inventive concepts, provided is method of making an implant device, comprising providing at least one spine, providing at least two c-rings, and using the spine to couple together the at least two c-rings.

In various embodiments, the method can further comprise maintaining the at least two c-rings in a substantially parallel orientation using the at least one spine.

In various embodiments, the method can further comprise forming ends of at least one c-ring overlap in a slip ring closure.

In various embodiments, the method can further comprise forming ends of at least one c-ring have a cut ring closure, wherein the ends do not overlap.

In various embodiments, the method can further comprise forming ends of at least one c-ring have mating teeth.

In various embodiments, the method can further comprise forming one or more of the at least two c-rings to be self-adjusting to accommodate a shape of the target location within the vessel of a body.

In various embodiments, the method can further comprise processing the one or more of the c-rings to have a memory shape that automatically expands when transitioned from a compressed state to a deployed state.

In various embodiments, the method can further comprise providing at least one stabilizer element extending from at least one c-ring.

In various embodiments, the method can further comprise forming the stabilizer element from a wire laterally extending from the at least one c-ring.

In various embodiments, the method can further comprise forming the stabilizer element in the shape of a zigzag.

In various embodiments, the method can further comprise providing at least one tissue penetration element configured to increase a frictional or mechanical resistance against inner walls of the vessel.

In various embodiments, the method can further comprise forming an end c-ring from the at least two c-rings to have a funnel-like shape.

In various embodiments, the method can further comprise forming the funnel-like shape to conform to an ostium or antrum of the vessel.

In various embodiments, the method can further comprise forming the at least two c-rings to have fingers disposed around a common central axis.

In various embodiments, the method can further comprise forming the at least one spine to include at least two helical ribbon spines that wrap at least partially around the common central axis.

In various embodiments, the method can further comprise forming the at least two helical ribbon spines bow out and away from the common central axis.

In various embodiments, the at least one spine can include a plurality of spines that couple together the at least two c-rings.

In various embodiments, the method can further comprise using two or more of the plurality of spines to couple together two c-rings.

In various embodiments, the method can further comprise providing a biologically inert coating to the implant device.

In various embodiments, the method can further comprise providing a coating to the implant device including one or more of drugs, biologics, chemicals, or combinations of one or more thereof.

In various embodiments, the method can further comprise providing a chemical ablation reagent coating to the implant device.

In various embodiments, the method can further comprise forming the at least one spine and the at least two c-rings from a single sheet of material and then shaping the material into the implant device form.

In various embodiments, the method can further comprise forming the implant device to deliver a force against tissue of the vessel in a range of between about 0.5 g/mm2 and about 300 g/mm2.

In various embodiments, the method can further comprising providing a micro-circuit in or on the at least one spine and/or at least one of the at least two c-rings that is configured to measure or monitor a value of electrical conduction propagating along the axis of a mammalian vessel within which the implant device is deployed.

In various embodiments, the method can further comprise disposing one or more electrodes in or on the at least one spine and/or at least one of the at least two c-rings that are in communication with the micro-circuit, the one or more electrodes configured for sensing conditions within the vessel and/or delivering energy to the vessel.

In various embodiments, the one or more electrodes can include ablation electrodes, mapping electrodes, or ablation and mapping electrodes.

In various embodiments, the micro-circuit can be configured to use an Ionic exchange with the vessel to charge a battery of the micro-circuit.

In various embodiments, the micro-circuit can be configured to measure and/or monitor a value of electrical conduction propagating along the axis of the vessel.

In various embodiments, the micro-circuit can be further configured to wirelessly transmit an indication of the electrical conduction in mammalian.

In various embodiments, the micro-circuit can be configured to receive an electromagnetic signal and to inductively heat the vessel in response to the electromagnetic signal.

In accordance with another aspect of the inventive concepts, provided is a method for treating a malady in a mammalian, comprising: determining a radial diameter of a vessel of a mammalian; providing an implant device having an unconstrained radial diameter that is greater than a radial diameter of a target location of the vessel. The implant device comprises at least one spine and at least two c-rings coupled together by the at least one spine, wherein the two or more c-rings are configured to radially expand from a compressed to a deployed state. The method also includes delivering the implant device to the target location and expanding the implant device at the target location, thereby dilating the vessel at the target location.

In various embodiments, the at least two c-rings can be maintained in a substantially parallel orientation by the at least one spine.

In various embodiments, ends of at least one c-ring can overlap in a slip ring closure.

In various embodiments, ends of at least one c-ring can have a cut ring closure, wherein the ends do not overlap.

In various embodiments, ends of at least one c-ring can have mating teeth.

In various embodiments, the method can further comprise one or more of the at least two c-rings self-adjusting to accommodate a shape of the target location within the vessel of a body.

In various embodiments, the method can further comprise the implant device delivering a force against tissue of the vessel in a range of between about 0.5 g/mm2 and about 300 g/mm2.

In various embodiments, one or more of the c-rings can have a memory shape and automatically expands when transitioned from the compressed state to the deployed state.

In various embodiments, the implant device can further comprise at least one stabilizer element coupled to at least one c-ring.

In various embodiments, the stabilizer element can be a wire laterally extending from the at least one c-ring.

In various embodiments, the method can further comprise stabilizer element can be a wire formed in the shape of a zigzag.

In various embodiments, the implant device can further comprise at least one tissue penetration element thereby increasing a frictional or mechanical resistance against inner walls of the vessel.

In various embodiments, the at least two c-rings includes an end c-ring can have a funnel-like shape.

In various embodiments, the funnel-like shape can conform to an ostium or antrum of the vessel.

In various embodiments, the at least two c-rings can have fingers disposed around a common central axis.

In various embodiments, at least one spine can include at least two helical ribbon spines that wrap at least partially around the common central axis.

In various embodiments, the at least two helical ribbon spines can bow out and away from the common central axis.

In various embodiments, the at least one spine can include a plurality of spines that couple together the at least two c-rings.

In various embodiments, two or more of the plurality of spines can couple together two c-rings.

In various embodiments, the implant device can further comprise a biologically inert coating.

In various embodiments, the implant device can further comprise a coating including one or more of drugs, biologics, chemicals, or combinations of one or more thereof.

In various embodiments, the implant device can further comprise a coating including a chemical ablation reagent.

In various embodiments, the at least one spine and the at least two c-rings can be formed from a single sheet of material.

In various embodiments, the method can further comprise the implant device delivering a radial force against tissue of a mammalian vessel at a target location that is sufficient to cause necrosis or apoptosis in the tissue in a deployed state, the necrosis or apoptosis sufficient to or delay electrical, neurological signal conduction traveling along an axis of the vessel and/or within an adjacent chamber in mammalian.

In various embodiments, the method can further comprise the implant device delivering a radial force against tissue of a mammalian vessel at a target location that is sufficient to compress at least one Ion channel in the adjacent tissue sufficient to delay electrical or neurological signals traveling along an axis of the vessel and/or within an adjacent chamber in mammalian.

In various embodiments, the implant device can further comprise a micro-circuit, the method further comprising using the micro-circuit, measuring and/or monitoring a value of electrical conduction propagating along the axis of a mammalian vessel within which the implant device is deployed.

In various embodiments, the implant device can further comprise one or more electrodes disposed in or on the at least one substrate and in communication with the micro-circuit, the method further comprising the one or more electrodes sensing conditions within the vessel and/or delivering energy to the vessel.

In various embodiments, the one or more electrodes can include mapping electrodes, the method further comprising mapping activity or geometry of a chamber associated with the vessel.

In various embodiments, the one or more electrodes can include ablation electrodes, the method further comprising ablating tissue at the target location.

In various embodiments, the method can further comprise the micro-circuit using an Ionic exchange with the vessel to charge a battery of the micro-circuit.

In various embodiments, the method can further comprise the micro-circuit measuring and/or monitoring a value of electrical conduction propagating along the axis of the vessel.

In various embodiments, the method can further comprise the micro-circuit wirelessly transmitting an indication of the electrical conduction in mammalian.

In various embodiments, the method can further comprise the micro-circuit receiving an electromagnetic signal and in response inductively heating the vessel at the target location.

### BRIEF DESCRIPTION OF THE DRAWINGS

The present invention will become more apparent in view of the attached drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the invention. In the drawings:

FIG. 1 is a perspective view of an embodiment of an implant device, according to aspects of the present invention;

FIG. 2A is a front view of an embodiment of an implant device with its ends not overlapped, according to aspects of the present invention;

FIG. 2B is a front view of the implant device of FIG. 2A with its ends overlapped, according to aspects of the present invention;

FIG. 2C is a front view of another embodiment of an implant device with tapered ends overlapped, according to aspects of the present invention;

FIG. 20 is a front view of another embodiment of an implant device with mating stepped shapes ends overlapped, according to aspects of the present invention;

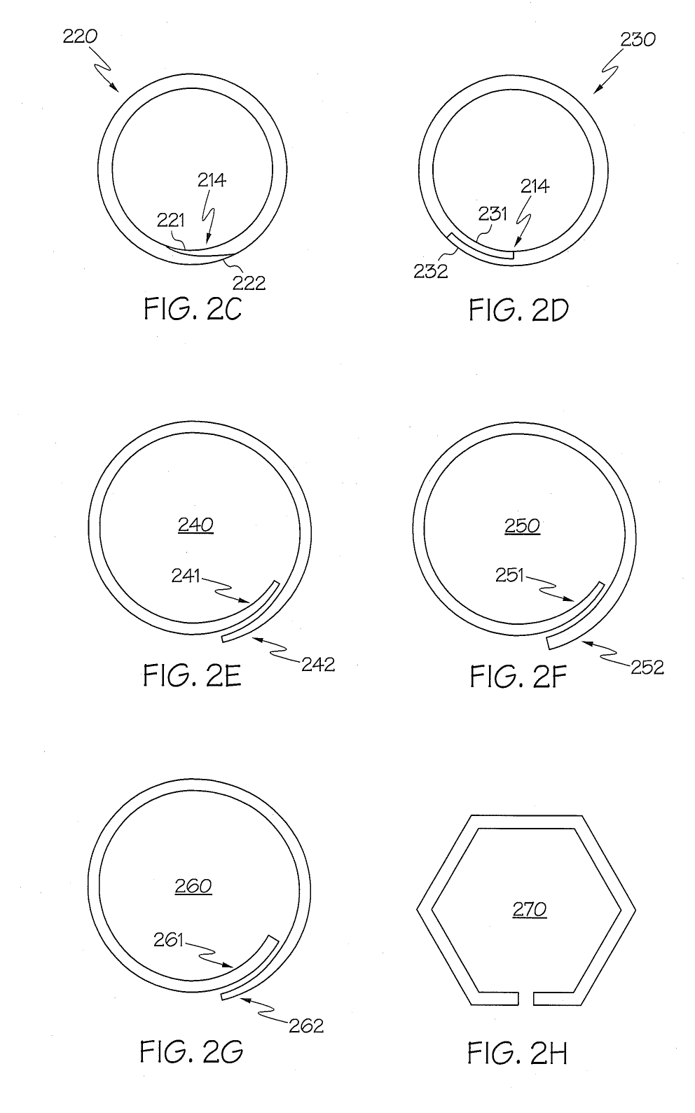

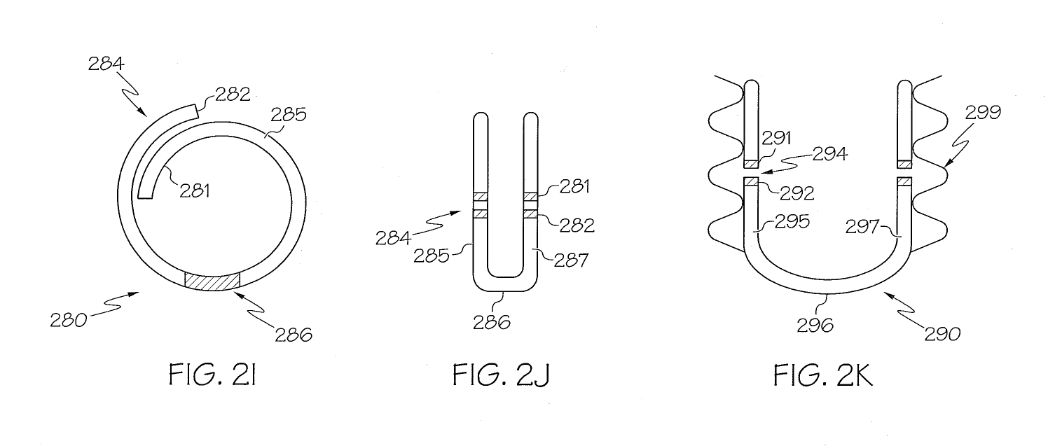

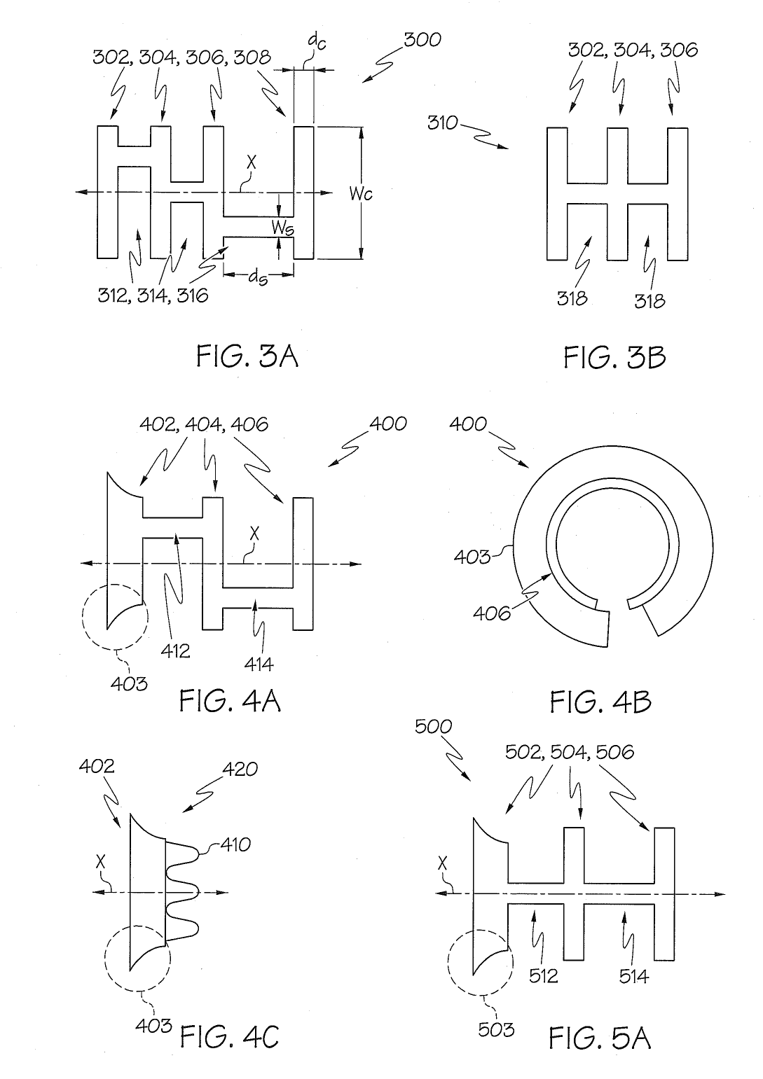

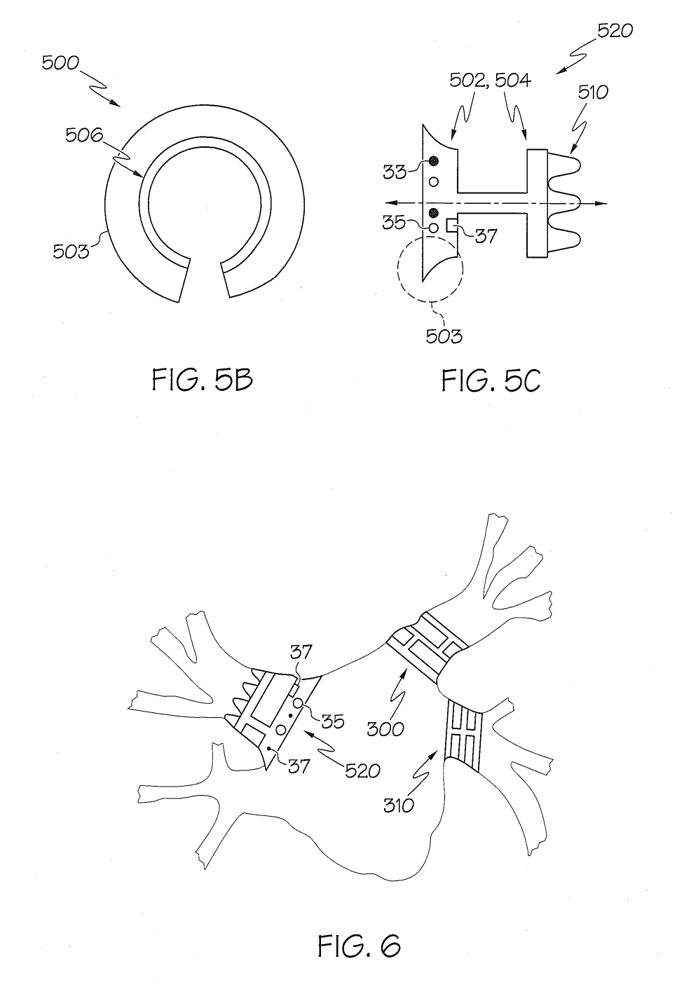

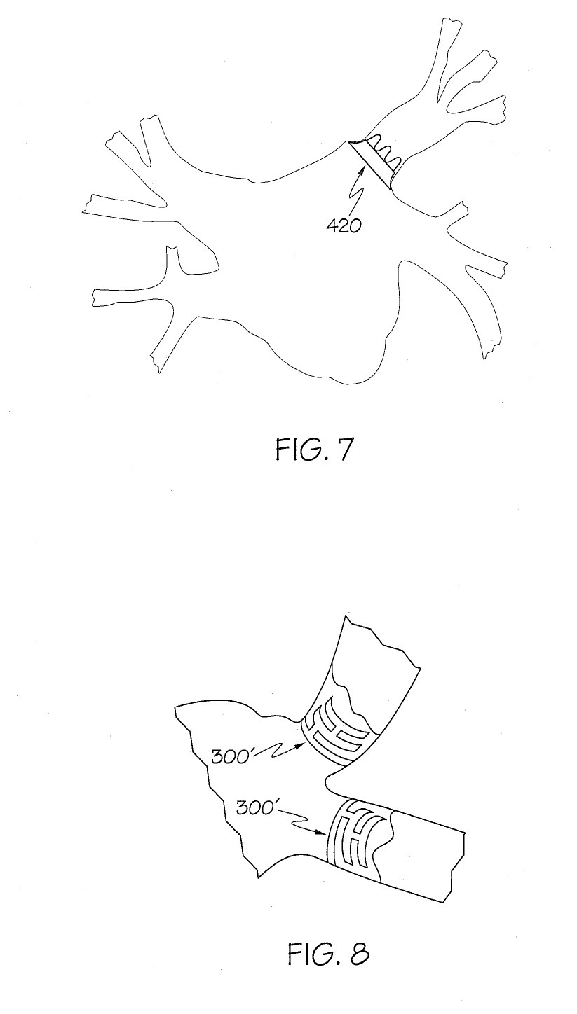

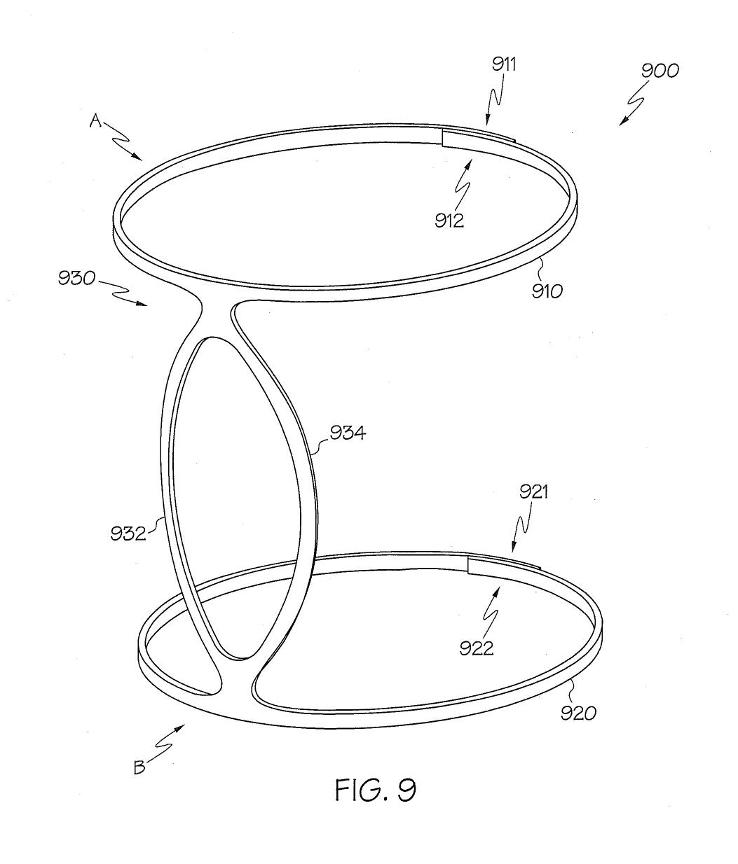

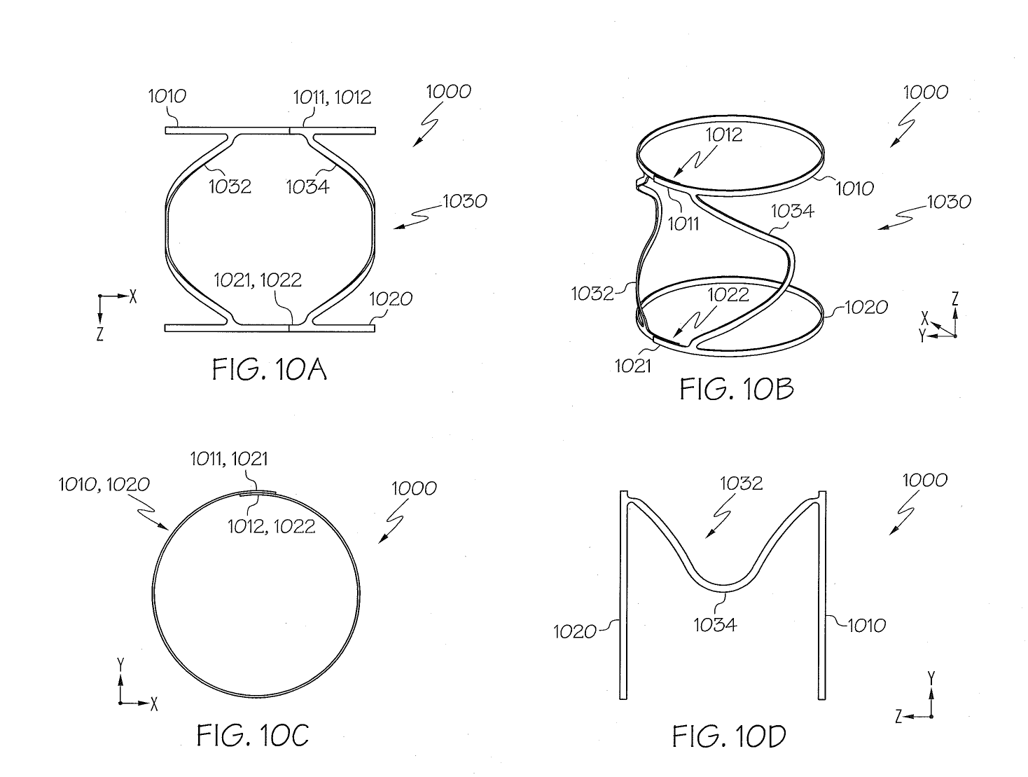

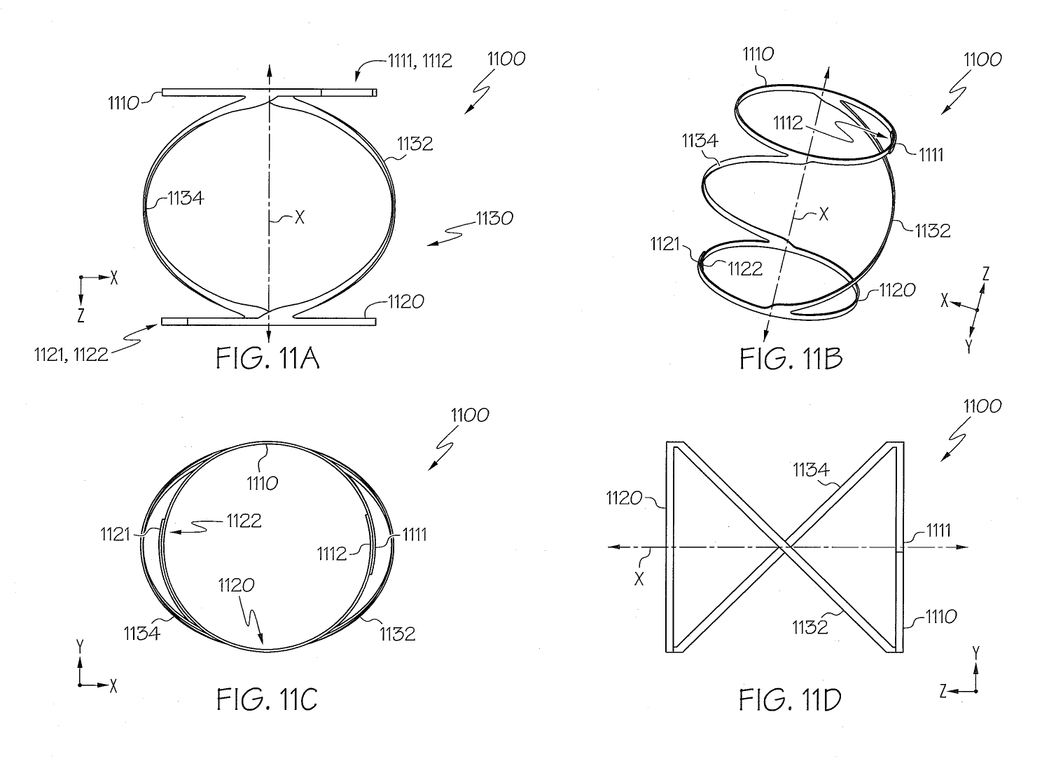

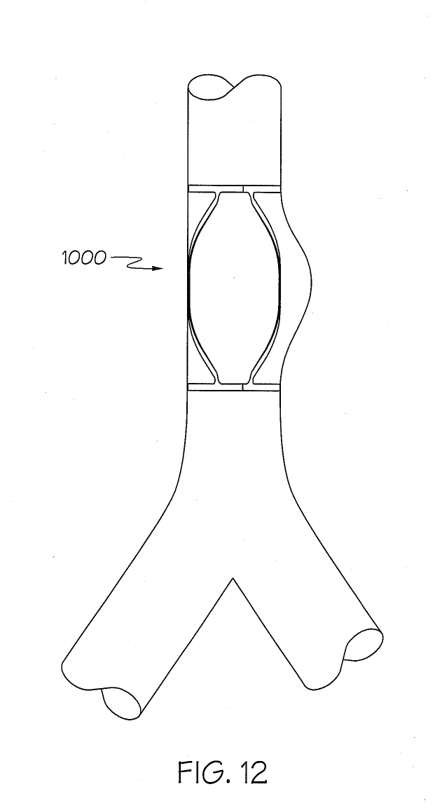

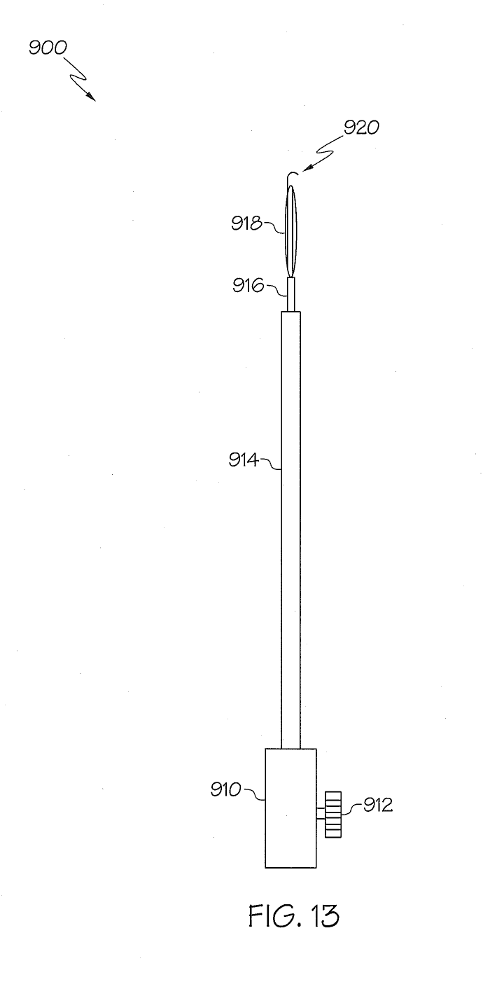

FIG. 2E is a front view of another embodiment of an implant device with partially tapered ends overlapped, according to aspects of the present invention;

FIG. 2F is a front view of another embodiment of an implant device with an inner tapered and an outer untapered end overlapped, according to aspects of the present invention;

FIG. 2G is a front view of another embodiment of an implant device with an inner untapered and an outer tapered end overlapped, according to aspects of the present invention;

FIG. 2H is a front view of another embodiment of an implant device having a hexagonal shape, according to aspects of the present invention;

FIG. 2I is a front view of another embodiment of an implant device having a a c-ring with overlapping ends and spine, according to aspects of the present invention;

FIG. 2J is a side view of another embodiment of an implant device having a spine coupling two cut rings, according to aspects of the present invention;

FIG. 2K is a side view of another embodiment of an implant device having a spine coupling two cut rings, each having a respective stabilizer element, according to aspects of the present invention;

FIG. 3A is a side view of an embodiment of an implant device having a plurality of c-rings connected by a plurality of spines, according to aspects of the present invention;

FIG. 3B is a side view of another embodiment of an implant device having a plurality of c-rings connected by at least one spine, according to aspects of the present invention;

FIG. 4A is a side view of another embodiment of an implant device having a flared c-ring, according to aspects of the present invention;

FIG. 4B is a front view of the implant device of FIG. 4A, according to aspects of the present invention;

FIG. 4C is a side view of another embodiment of an implant device having a flared c-ring and stabilizer element, according to aspects of the present invention;

FIG. 5A shows a side/top view of another embodiment of an implant device having multiple c-rings, including a flared, that are connected by aligned spines, in accordance with aspects of the present invention;

FIG. 5B shows a front view of implant device of FIG. 5A, according to aspects of the present invention;

FIG. 5C shows a side/top view of yet another embodiment of an implant device 520, having a flared c-ring, stabilizer element, and mapping electronics, in accordance with aspects of the present invention;

FIG. 6 shows an embodiment here a plurality of implant devices is deployed within a mammalian body, in accordance with aspects of the present invention;

FIG. 7 shows an embodiment where the implant device of FIG. 4C is deployed within a mammalian body, in accordance with aspects of the present invention;

FIG. 8 shows another embodiment here a plurality of implant devices is deployed within a mammalian body, in accordance with aspects of the present invention;

FIG. 9 provides a perspective view of another embodiment of an implant device, according to aspects of the present invention;

FIGS. 10A through 10D show four views of another embodiment of an implant device, according to aspects of the present invention;

FIGS. 11A through 11D show four views of another embodiment of an implant device, according to aspects of the present invention;

FIG. 12 is a schematic view of an implant device deployed within a mammalian body, in accordance with aspects of the present invention; and

FIG. 13 is a side view of an embodiment of an implant device delivery system, according to aspects of the present invention.

### DETAILED DESCRIPTION OF PREFERRED EMBODIMENTS

Various exemplary embodiments will be described more fully hereinafter with reference to the accompanying drawings, in which some exemplary embodiments are shown. The present inventive concept may, however, be embodied in many different forms and should not be construed as limited to the exemplary embodiments set forth herein.

It will be understood that, although the terms first, second, etc. are be used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present invention. As used herein, the term “and/or” includes any and all combinations of one or more of the associated listed items.

It will be understood that when an element is referred to as being “on” or “connected” or “coupled” to another element, it can be directly on or connected or coupled to the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in the figures is turned over, elements described as “below” and/or “beneath” other elements or features would then be oriented “above” the other elements or features. The device may be otherwise oriented (e.g., rotated 90 degrees or at other orientations) and the spatially relative descriptors used herein interpreted accordingly.

In accordance with aspects of the present invention, provided is an implant device that is configured and sized to be permanently or temporarily implanted in a mammalian body, such as in a blood vessel (e.g., artery, vein) or organ. For example, a device that is permanently implanted can be placed within a body indefinitely, e.g., years, such as for the remainder of life of the mammalian or until the device needs replacing or is no longer needed. A device that is temporarily implanted into a body can be placed within the body for seconds, minutes, hours, days, or months, or other time, with an intention or plan for removal after the occurrence of one or more predetermined times, tasks, or events. For instance, such a device could be implanted to aid in a diagnostic or treatment procedure, and removed during or at that the conclusion thereof.

An implant device in accordance with aspects of the present invention can include one or more c-rings and one or more spines, wherein spines can connect c-rings. The term “c-ring” for these purposes is a structural element at least partially surrounding an open center. In presently preferred embodiments, the ends of the c-rings are not rigidly connected at its ends. In fact, the size of the c-ring may be adaptable, such the ends of the c-ring experience relative movement when deployed as part of such adjustment. The c-ring may be considered to include fingers extending from at least one spine, around an axis of the implant device, which could be coincident with an axis of a vessel within which the implant device is implanted.

Within an implant device, one or more spines can be connected to at least one c-ring to provide overall stability to the implant device. The spine of an implant device can comprise at least one structural member that connects at least two c-rings. The spine can server to maintain a separation of the c-rings—at least at the connection points. In some embodiments, a spine can be used to maintain two c-rings at a substantially fixed distance apart, wherein the c-rings could be maintained in a parallel orientation or at some other relative angle or orientation. However, in other embodiments, one or more c-rings coupled to a spine may be configured to move along the spine to adjust a separation distance between the c-rings. A spine can be a straight or substantially straight member, or could be curved or angled with respect to at least one c-ring and/or a central axis of the implant device. Where an implant device includes more than one serially arranged c-rings, the spines could be substantially collinear or offset. In some embodiments, more than one spine can be used to connect two c-rings. In various embodiments, spines could be straight, curved, or take some other shape or pattern. Generally, the spines and c-rings can be arranged in many different patterns and configurations, depending on the malady, treatment site, or both, or other factors, such as cost, manufacturing, and/or reliability considerations.

In various embodiments, a c-ring may take a variety of shapes, e.g., a c-ring could be substantially round, oval, octagon, hexagon or any of a variety of other shapes, or combination of such shapes. The c-rings and/or its fingers may vary in one or more of thickness, width, stiffness, length, and so on. In some embodiments, a c-ring can have concave or convex end shape, e.g., at a proximal and/or distal end of the implant device. Such a shape could be configured to enable the implant device to conform to the ostium of a vessel. For instance, the c-ring may have an inner edge diameter and an outer edge diameter that are not equal, in some embodiments. The implant device may employ a single c-ring with this concave or convex shape for apposition to said tissue of an ostium of a vessel, such as a pulmonary vein, in some embodiments.

In some embodiments, the fingers of a c-ring can stop just short of forming a closed ring, such as in the case of the basic formation of a c-ring, or may include fingers that pass each other, e.g., overlap. In the latter case, the ends of the c-ring of the implant device may be slip fit, lap jointed or otherwise configured so that the ends of the implant device are unconnected and free to move in a circumferential or radial direction, e.g., to radially expand or diminish. Such movement can be along the same plane as the distal end of the implant or off-plane from the distal end of the implant device. In some embodiments, therefore, the fingers of the c-ring need not share the same plane. For example, the fingers could be offset from each other, e.g., with one finger passing by a side of and/or above the other finger. In such cases, the ends of the fingers that form the c-ring may include features to enable self-adjustment or user controlled adjustment to accommodate a target vessel or tubular structure. As an example, the ends of a c-ring can incorporate a ratchet-style interaction to lock-in an adjustment.

In various other embodiments, fingers of a c-ring may meet at their ends. In such cases, the ends of the fingers of said c-ring may share a similar plane and touch (or nearly touch) each other such that from an end view it appears that the fingers are connected, e.g., as a connected concentric ring. In this sense, the ends may be abutted (or nearly abutted). In some embodiments, ends of the fingers could be formed like teeth that close together, whether fully or partially.

The implant device may optionally include a stabilization element connected to one or more c-rings or a spine of the implant device, to ensure a minimum of migration and implant stability. The stabilizer element could be made of a wire, which could be configured in a zigzag pattern. The wire could be connected to an edge of the c-ring. In some cases, more than one stabilizer element can be connected to or form part of a c-ring. In other embodiments, the stabilizer element could take other forms, not necessarily a shaped wire. In various embodiments, other forms of tissue penetration elements could be coupled to or form part of a spine, c-ring, or both, e.g., barbs. Such tissue penetration elements could be scalloped, or have another shape configured to increase frictional or mechanical resistance against movement and enable a locking mechanism to adjoin the ends of the implant device. Thus, in some embodiments, the ends of the fingers that form the c-ring may employ features for sizing and holding position in a tubular or circumferential shape, such as the locking mechanisms, e.g., interacting teeth with ratcheting or similar mechanical devices along the ends of the finger that form the c-rings.

It is noted that limiting migration is assisted by the shape and structure of the implant device. In particular, the overall structure of the implant device ensures that a longitudinal force, along the axis of the device, tends to be absorbed by a compression of the implant during deployment, similar to the way in which a spring compresses, although the construction ensures that the spring constant may be extremely low, especially in the planner direction. This may be contrasted with other more stent-like structures, which are designed such that a longitudinal force is transmitted along the typical chain link or honeycomb structure, causing translation or a change of radius of such structures rather than compression.

In addition, an external surface of the implant device may have a textured surface, or may include a polymer sleeve, ePTFE, PTFE, Dacron or a combination of these materials, to further aid the device in fixation to the tissue. Such polymer sleeve may also include a microcircuit to wirelessly enable electrogram interpretation during and after a procedure. Furthermore, a coating or biological agent of the implant surface may be employed to further reduce migration and/or erosion of the implant device, and thereby minimize neointimal hyperplasia, reduce clotting, or support or aid in reducing AF burden, neurological disorders or other maladies.

In various embodiments, the fingers of the c-ring may be formed from a flat sheet or ribbon or material, or a combination of these. In some embodiments, the entire implant device can be formed out of a single thin sheet of material, which may then be processed or formed into the generally tubular form. The implant design and processing may also include elements for assembly, such as lamination, heat setting, or other joining designs or approaches.

In various embodiments, the fingers may have a width substantially greater than a thickness of said finger. A spine can have a width substantially greater than a thickness of said spine. As an example, in a particular implant device, a spine can have a thickness greater than 0.1 mm and at least one c-ring can have a thickness greater than 0.1 mm. In various embodiments, the implant device could have at least one metal spine and polymer c-ring, or vice versa, or any combination of such. The implant device may have a diameter in a range of about 0.5 mm to 70 mm, as an example. The implant device may also include micro-circuits for wireless communication from within a mammalian body to an external source or system outside the mammalian body.

The implant device, with spine and c-rings, may be formed from a biocompatible metal, alloy, biocompatible polymers or elastomers, bio-absorbable polymer or any combination of these materials, such as a spine formed from a polymer and the c-rings formed from an alloy or a metal, e.g., a super-elastic alloy, e.g., Nitinol. Such material may exhibit useful shape memory properties. The implant device may be made by cutting the device, or portions thereof, form a sheet of material, wherein the sheet of material can be machined, etched, stamped, laser cut or injection molded or a combination of these methods to form a substantially flat implant device. The flat workpiece is then shaped into the desired configuration. For example, an implant device may be formed from a sheet of Nitinol that is laser cut and then shaped to form spines and c-rings. For implants made from ribbon, wires, exemplary values of the ribbon width may be, e.g., 1-4 mm, e.g., between 0.5 and 10.5 mm.

If the implant device is made of materials that are bio-absorbable, then the same may eventually be absorbed into the body by virtue of the endothelialization, leaving only (and at most) a scar visible on the inside of the vessel or chamber.

While not required in all embodiments, various coatings, coverings or other agents may be applied or made part of the implant device, e.g., spine, and/or c-ring, or other elements of said implant device. Such coatings or agents may be capable of otherwise treating a malady or promoting the healing process. Such coatings may include drugs, biologics, chemicals, or combinations, and the same may cause some degree of necrosis that, by itself or in combination with the mechanical compression, can act as a treatment. For example, a coating including alcohol may be employed as a sort of chemical ablation reagent. Such coatings may also enhance endothelialization, as discussed above. As another example, the implant device may be coated with gold, platinum, tantalum, e.g., a coating of at least 3-5 microns.

In various embodiments, an implant system includes an implant device and a delivery catheter, wherein the implant device includes at least one spine and at least one c-ring and, optionally, other elements described herein. The delivery catheter can be configured to hold the implant device at a distal end, and then deploy the implant device when delivered to a target location using the catheter. A distal delivery end of the catheter can be expandable, as with a balloon or similar catheter, wherein the balloon (or other expansion device) can deploy the implant device at the desired location within a vessel. Using such deliver catheter, the implant device can be delivered and deployed within a chamber, appendage, or vessel of a mammalian body, such as a pulmonary vein, as illustrated schematically in FIGS. 6-8 and 11B. Other methods could also be used to deploy the implant device, such as more invasive surgical methods.

In various embodiments, c-rings may be biased in the unconstrained state, put into a compressed state for delivery into the body, and then, once at the intended delivery site, unconstrained for expansion within a blood vessel or organ. In such a case, the expansion will typically be limited by the blood vessel or organ tissue at the delivery site. The spine with c-ring may be designed to deliver a force against the tissue sufficient to prevent migration of said implant. Furthermore, the forces against tissue may also include a range of between about 0.5 g/mm² and about 300 g/mm², as an example. The delivery mechanism of the implant device can be, for example, a catheter.

A diameter of the un-deployed implant device may be in a range of about 0.5 mm to 80 mm, as an example. The diameter of the deployed implant device may be about 2 mm to 80 mm after deployment at the delivery site is completed. Alternatively, an asymmetric pattern may be employed. Using these values, a c-ring when un-deployed may be significantly oversized compared to the vessels for which they are intended. They may be, e.g., oversized by 10-100%, or more particularly in a range of about 20-60%.

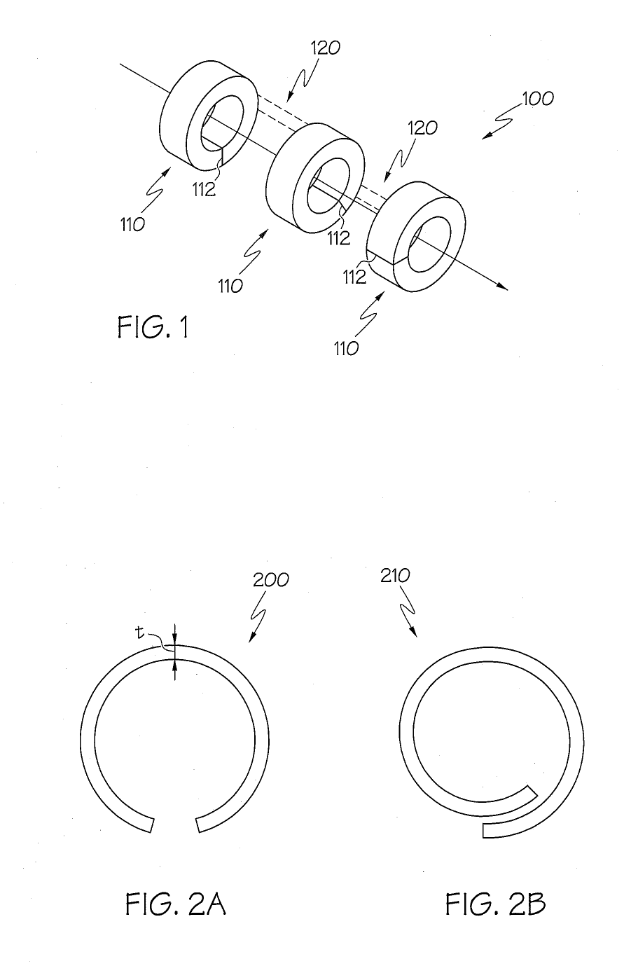

FIG. 1 is a perspective view of an embodiment of an implant device 100, according to aspects of the present invention. The implant device 100 is a generally elongate device disposed around a central axis (X), but the implant device is preferably not a stent tube. Rather, the implant device can be considered one or more c-rings coupled together by a stabilizing spine. Here, the implant device 100 include three c-ring 110 and two spines 120, which couple together the c-ring 120. In other embodiments, a different number of c-rings and/or spines could be used. In this embodiments, spines 120 are not collinear, but in other embodiments they could be collinear. In the embodiment of FIG. 1, a c-ring is a ring-type structure that has discrete ends. More particularly, in the embodiment of FIG. 1, each of the c-rings 110 has its discrete ends abutted together to form a closure 112. In other embodiments, the ends could have a different arrangement, as discussed above.

In FIG. 1, the three c-rings 120 are arranged in parallel. The closures 112 of the c-rings are angularly offset in FIG. 1, but need not be in other embodiments. That is, in various embodiments, the closures can be aligned or collinear.

FIG. 2A is front and/or rear views of an embodiment of a c-ring 200 that can be used to form at least a portion of an implant device, in accordance with aspects of the present invention. C-ring 200 is similar to c-ring 110 shown in FIG. 1, in that it readily lends itself to abutting the ends, if not to the point of contact, then at least nearly in contact.

The c-rings will have a thickness (t). The value of t can depend on the material used for making the c-ring and the forces to be applied to the c-ring, particularly at its intended deployment location, i.e., the delivery site.

FIG. 2B shows front and/or rear views of another embodiment of a c-ring 210 that can be used to form at least a portion of an implant device, in accordance with aspects of the present invention. C-ring 210 has its ends overlap, which can be useful in accommodating different size tissue openings. It can also lend itself to more easily being rolled up and constrained for insertion into a body, e.g., via a catheter, and then deployed and unconstrained at a delivery site within the body, e.g., in a blood vessel or organ.

In the embodiment shown in FIGS. 2A and 2B, the c-ring has a substantially uniform or constant thickness from one (first) end to the other (second) end, but this need not be the case in all embodiments. If the first and second ends of the c-ring are abutted when deployed at a delivery site within a body, the c-ring can maintain a substantially uniform thickness, as is shown in FIG. 1. Or, at least, the abutting arrangement would represent the smallest diameter available for a c-ring, but a larger diameter could be achieved by separating the ends of the c-ring.

However, if the first and second ends of the c-ring are overlapped when deployed at a delivery site within a body, the c-ring having a substantially uniform thickness from the first end to the second end, as is shown in FIG. 2B, will not have a substantially smooth inner surface when deployed. The overlap could create a protrusion into the path of fluid flow through the implant device. The smaller the thickness t, the less the protrusion.

FIGS. 2C and 2D show two different embodiments where and overlapping portions of the ends of a c-ring can be narrower than other parts of the c-ring to reduce the overall thickness at a closure 214, e.g., by tapering or stepping down the thickness. Therefore, in some embodiments, the thickness of the ends of the c-ring could be formed such that when overlapped, in its final delivered configuration, the c-ring has a substantially smooth inner surface, e.g., again, as in FIGS. 2C and 2D. Here, the overall thickness of the c-ring can remain about the same, even at the closure. Maintaining a substantially smooth inner surface can reduce the possibility or degree of turbulence in the fluid flow through the implant device, which can be advantageous for the mammalian and short and/or long term effectiveness of the implant.

FIG. 2C shows front and/or rear view of another embodiment of a c-ring 220 that can be used to form at least a portion of an implant device, in accordance with aspects of the present invention. C-ring 220 has its ends 221, 222 overlapped, which can be useful in accommodating different size openings. It can also lend itself to more easily being rolled up and constrained for insertion into a body, e.g., via a catheter, and then deployed and unconstrained at a delivery site within the body, e.g., in a blood vessel or organ.

In the embodiment of FIG. 2C, the first and second ends of the c-ring are tapered to maintain a substantially smooth inner surface within the implant device.

In the embodiment of FIG. 2D, the first and second ends 231, 232 of the c-ring 230 have corresponding and mating stepped shapes which can maintain a substantially smooth inner surface within the implant device when deployed.

FIG. 2E shows front and/or rear view of another embodiment of a c-ring 240 that can be used to form at least a portion of an implant device, in accordance with aspects of the present invention. C-ring 240 has its ends 241, 242 overlapped, which can be useful in accommodating different size openings. Here, both ends 241 and 242 are thinned, so they have a smaller thickness that the other portions of the c-ring 240. Therefore, an overlap of ends 241, 242 would create less of a protrusion into a fluid path. In some embodiments, the combined thickness of ends 241, 242 can be the same or about the same as a thickness of the remaining portions of the c-ring 240. This shape can also lend itself to more easily being rolled up and constrained for insertion into a body, e.g., via a catheter, and then deployed and unconstrained at a delivery site within the body, e.g., in a blood vessel or organ.

FIG. 2F shows front and/or rear view of another embodiment of a c-ring 250 that can be used to form at least a portion of an implant device, in accordance with aspects of the present invention. C-ring 250 has its ends 251, 252 overlapped, which can be useful in accommodating different size openings. Here, one end 251, an internal end, is thinned, reducing the thickness of the overlapped portion. Therefore, an overlap of ends 251, 252 would create less of a protrusion into a fluid path. This shape can also lend itself to more easily being rolled up and constrained for insertion into a body, e.g., via a catheter, and then deployed and unconstrained at a delivery site within the body, e.g., in a blood vessel or organ.

FIG. 2G shows front and/or rear view of another embodiment of a c-ring 260 that can be used to form at least a portion of an implant device, in accordance with aspects of the present invention. C-ring 260 has its ends 261, 262 overlapped, which can be useful in accommodating different size openings. Here, one end 262, an external end, is thinned, reducing the thickness of the overlapped portion. Therefore, an overlap of ends 261, 262 would create less of a protrusion into a fluid path. This shape can also lend itself to more easily being rolled up and constrained for insertion into a body, e.g., via a catheter, and then deployed and unconstrained at a delivery site within the body, e.g., in a blood vessel or organ.

In other embodiments, the c-ring need not be circular, e.g., it could be pentagonal, hexagonal, octagonal, nonagon, decagon and so on. In some embodiments, the c-ring could have an oval shape. In other embodiments, the c-ring could have an irregular shape. FIG. 2H shows a c-ring 240 having a hexagonal shape, as an example.

FIG. 2I shows front and/or rear view of another embodiment of an implant device 280, in accordance with aspects of the present invention. Implant device 280 has a c-ring 285 has its ends 281, 282 overlapped at a closure 284, similar to the embodiment of FIG. 2B. A spine 286 is shown as being integral with a portion of the c-ring 285. This shape can also lend itself to more easily being rolled up and constrained for insertion into a body, e.g., via a catheter, and then deployed and unconstrained at a delivery site within the body, e.g., in a blood vessel or organ.

FIG. 2J shows an embodiment of a side view of the implant device 280, which is a variation of that shown in FIG. 2I, in accordance with aspects of the present invention. The implant device 280 includes spine 286 coupling two rings 285, 287. Here, rings 285, 287 are cut rings, having non-overlapping ends 284, as in FIG. 2A.

FIG. 2K shows another embodiment of a side view of the implant device 290, which is a variation of that shown in FIG. 2J, in accordance with aspects of the present invention. The implant device 290 includes spine 296 coupling two rings 295, 297. Here, rings 295, 297 are cut rings having an opening 294 between non-overlapping ends 291, 292, as in FIG. 2A. Additionally, each of the rings 285, 287 includes a respective stabilizer element 299, which helps stabilize the implant device, and resist migration forces.

In FIG. 3A, a top view of an embodiment an implant device 300 is shown, and includes a plurality of substantially laterally (or cross-wise) disposed c-ring 302, 304, 306, and 308, such as c-ring 200, 210, 220, 230, and 240 of FIGS. 2A through 2E. The c-rings are coupled together by one or more spines, providing stability to the implant device. In this embodiment, the spines are substantially longitudinally disposed spines 312, 314, and 316.

C-ring 302, 304, 306, and 308 are shown having substantially the same width (wc), but this need not be the case in all embodiments. That is, in some embodiments, one or more of a plurality of c-rings can have different widths within the same implant device.

Spines 312, 314, and 316 are shown to have substantially the same width (ws), but this need not be the case in other embodiments. That is, in some embodiments, one or more of a plurality of spines can have different widths within the same implant device. Additionally, spines 312, 314, and 316 are shown as being generally straight and at right angles to the c-rings in this embodiment, but in other embodiments the spines could be at an angle of less than or greater than 90 degrees with respect to the c-rings, and/or could be other than straight, e.g., be curved or include one or more curved portions or one or more straight portions, or combinations thereof. Furthermore, different spines can be disposed between the same two c-rings and/or between different pairs of c-rings.

In FIG. 3B, another embodiment of an implant device 310 is shown, as an example. In this embodiment, three c-rings 302, 304, and 306 are aligned in parallel and two spines 318 are shown joining the 3 c-rings. The spines are arranged to be substantially collinear.

FIG. 4A shows a top view of another embodiment of an implant device 400, in accordance with aspects of the present invention. In FIG. 4A, there are provided three c-rings 402, 404, and 406 and at least two spines 412 and 414. In this embodiment, c-rings 404 and 406 are similar in structure, each having a flat outer surface 405, 407. For example, the outer surfaces of c-rings 404 and 406 may be in parallel. The at least two spines 412 and 414 are offset from each other.

However, an outer surface 403 of c-ring 402 is flared in this embodiment, with the flare widening as it extends external to the implant device, and other c-rings. The flared shape can be useful, for example, when the deliver site tissue has a similarly flared opening. In this embodiment, therefore, the implant device 400 has some portions that are wider than other portions.

FIG. 4B shows a front view of implant device 400, where the flared outer surface 403 of c-ring 402 is apparent.

FIG. 4C shows a top view of yet another embodiment of an implant device 420, in accordance with aspects of the present invention. In FIG. 4C, provided is a flared c-ring 402, as in FIG. 4A with a wire stabilizer element 410, in a zigzag shape. The zigzag wire stabilizer element 410 provides stability to the c-ring within the blood vessel or organ within which it is delivered.

FIG. 5A shows a top/side view of another embodiment of an implant device 500, in accordance with aspects of the present invention. In FIG. 5A, there are provided three c-rings 502, 504, and 506 and at least two spines 512 and 514. In this embodiment, c-rings 504 and 506 are similar in structure, each having a flat outer surface 505, 507. For example, the outer surfaces of c-rings 504 and 506 may be in parallel. The at least two spines 512 and 514 are aligned with each other.

As in FIGS. 4A-4C, an outer surface 503 of c-ring 502 of FIG. 5A is flared in this embodiment, with the flare widening as it extends external to the implant device, and other c-rings. The flared shape can be useful, for example, when the deliver site tissue has a similarly flared opening. In this embodiment, therefore, the implant device 500 has some portions that are wider than other portions.

FIG. 5B shows a front view of implant device 500 of FIG. 5A, where the flared outer surface 503 of c-ring 502 is apparent.

FIG. 5C shows a top/side view of yet another embodiment of an implant device 520, in accordance with aspects of the present invention. In FIG. 5C, provided is a flared c-ring 502, as in FIG. 5A with a wire spine 512 coupling c-ring 502 to c-ring 504. There is a zigzag wire stabilizer element 510 coupled to an opposite end of c-ring 504.

FIG. 6 shows a depiction of a mammalian organ treated with different implant device embodiments, as an example of how such implant devices could be used. Here, implant devices 300, 310, and 520 are shown, for illustrative purposes.

Referring to the embodiments of FIGS. 5C and 6, an implant device 520 may include ablation device and/or a mapping device integral with or provide in conjunction with a proximal c-ring 502 and/or a distal c-ring 504. In particular, the distal ring 504 may incorporate a number of mapping electrodes 33. The proximal ring 502 may incorporate a number of ablating electrodes 35, or both types of electrodes could be located in either of the c-rings. The distal set may enter into a pulmonary vein and become temporarily apposed to the inner lumen therein. In this sense, the implant device 520 with two sets of electrodes 33, 35 may be disposed similarly to the implanted device discussed above, but in this case, the same could be retracted after treatment.

The distal ring employs its electrodes for mapping, while the proximal ring may employ its electrodes 33, 35 for mapping and/or ablation, in some embodiments. The apposed electrode of the distal c-ring may be as noted above, and while the same may become lodged with respect to translational displacement, the same may also be easily rotated with respect to a track formed by the pressure of the ring against the tissue of the pulmonary vein. The proximal ring electrodes may then contact the ostium and via RF ablation cause necrosis of a ring of tissue around the ostium. In this embodiment, one of the c-rings can include a micro-circuit 37 coupled to the electrodes for monitoring and/or control thereof.

FIG. 7 shows another depiction of a mammalian organ treated with a different implant device embodiment, as an example of how such implant devices could be used. Here, implant device 420 is shown, for illustrative purposes.

FIG. 8 shows a depiction of a mammalian organ treated with a different implant device embodiment, as an example of how such implant devices could be used. Here, implant device 300′ is similar to implant device 300, and is shown for illustrative purposes.

As discussed above, in various embodiments, an implant device may be plated with conductive materials such as gold, platinum etc., bio-absorbable, temporary, permanent, removable, or the same may be configured and designed to be absorbed into the body after a period of time. In a removable embodiment, a removable portion (which may be the entire implant or a portion thereof) may be installed for a period of time, e.g., between 30 minutes and 24 hours, and then removed. While the procedure and device have been described in the context of the left atrial appendage ligation, the same may be conveniently employed in other areas of the body to prevent or block the flow of a gas, liquid or other in a body. In addition, the device may be employed to invoke a neurological response of the ganglion plexus, sympathetic nervous system and other neurological conditions.

In some embodiments, the implant device may include one or more variations. The implant device may be implanted within an appendage or vessel of the heart and may generate sufficient compressive force to block the blood flow. Said implant device may have a material composition, surface treatment, coating, or biological agent and/or drug to cause a human biological response, e.g., intima hyperplasia or endothelization, in a controlled or semi-controlled way in order to affect a long-term appendage closure. The implant device may have at least one c-ring, circumferential ring or coil like structure, and indeed more, and may include at least one spine oriented distally from the outer diameter of the first c-ring or coil and terminating within the vessel to prevent migration of the implant. Said implant may be made of a round wire or ribbon profile that is shaped into a c-ring or coil or other shapes. The implant device may have various circular shapes such as octagon, pentagon, as discussed herein that are unconstrained meaning although said implant might be substantially round in fact the implant might be formed to take on a Hexagon, Octagon, or Pentagon shaped and be free-form such that the distal end of the device do not connect to the proximal end of the device only by the spine connecting the c-ring or coil or coils.

The spine may be straight or form various patterns, such as a helical pattern yet having a hexagon, octagon or other shape cross-sectional c-ring or coil shapes designed to focus mechanical force in a circumferential or helical compressive pattern toward the inner surface of a vessel, appendage or chamber. These shapes can include, but are not limited to, round, oval or circular, triangular, rectangular, “U”-shaped, or any number of other shape combinations. The implant may have a material composition alloys, polymers or combinations of alloys and polymers and/or geometry designed to sufficiently conform to tissue to prevent coagulation or thrombus, and may include a material coating to further reduce or prevent such coagulation or thrombus.

In some embodiments, the implant device may act as an electrical wave reflector, changing the course of the electrical wave back to its origin and in some implementations acting as a cancellation medium to electrical waves emanating from the source. Various embodiments of the implant device may be employed in combination with an ICD to deliver currents or voltages to heart tissues. Such devices may be coupled to an ICD in a wired or wireless modality. Other devices that may take advantage of the convenient placement of the implanted devices may similarly benefit from coupling to the same.

As examples, mechanisms of operation of the inventive implant device for various clinical treatment modalities include, but are not limited to, arrhythmias, hypertension, AAA, left atrial appendage closure, where the implant device can include a spine with c-ring or like device with tissue penetration elements. The implant device may be configured to work, in some respects, opposite a traditional implant device that is designed to prop open a vessel or chamber by exhorting a outward force, such as in the case of a stent providing a mechanical radial outward force to open or maintain the patency of a vessel or other body part that is generally constant along its tubular length. In various embodiments of this invention, the implant device includes at least one c-ring element affixed to a spine, wherein a c-ring using an inward force can be configured to close a vessel or structure in a mammalian body to reduce or prevent the passage of a gas, fluid or other in substance. The implant device may be constructed from a super elastic material or other material previously described in this document. The tissue penetration element can be designed to penetrate and hold the tissue, while the c-ring can retract using the inward mechanical force to pull inward the tissue as with in the use for left atrial appendage closure or other chambers or vessels within a mammalian body capable of closure sufficient to slow or block the flow of a gas, fluid or other substance from entering a targeted area or region in mammalian body.

It is believed that the amount of pressure or inward force necessary for clinical or other benefit should be more than about 0.5 grams per square millimeter, but less than 3000 grams per square millimeter. However, in general, it is believed that the amount of inward pressure needed will primarily be a function of the material used, the diameter of the appendage, thickness of said tissue, chamber size, artery or vein diameters, and thickness of the tissue.

It is noted that the implant device may remain inside the appendage, in the clinical field of use of left atrial appendage closure. The implant c-ring and tissue penetration element may perform an anchoring function as well as a conductive or neurological delay function. In some cases, merely a closing down of the appendage or other tissue may be enough to be clinically effective. For example, a 50% reduction in appendage patency may also be sufficient to block eherent electrical signals that cause arrhythmias or other maladies and be highly significant in stopping the propagation of thrombus formation. In any case, the implant device's geometry, roughly matching the shape of the targeted area, further enhances this effect. If the configuration of the implant device is such that these are disrupted, then the disruption can act as an efficacious treatment per se. It is also noted that the implant device inside the vessel, appendage or other allows for a therapeutic treatment modality, but without the serious complications associated with energy ablation catheters devices, such as coag and thrombus formation.

It is also noted that the implant device may cause the appendage in which it dwells to become more oval or round, or otherwise to maintain a more open shape than that which it adopted before, in the absence of the implant device. In some cases, the implant device may be specifically installed to perform the function of a ligation device blocking blood flow in major vessels or organs.

The biological response of endothelization cell proliferation is designed to enclose the entrance to said appendage by use of biological, endothelial cells proliferation. The treatment of the implant device refers to, e.g., the level to which the device has been roughened so as to act as an irritant to the adjoining tissue. The amount of endothelialization may be ‘tuned’ by this degree of roughening, alloy mixtures such as increased copper content or other materials. The treatment may also be via surface modification, coatings, coverings or the like.

In some embodiments, a metallic nature of the implanted device may be employed to provide a level of active heating so as to heat or necrose tissue adjoining or surrounding the implant. For example, such heating may be by way of inductive heating, chemical, chemical reaction, or chemical reaction, as examples, within blood in mammalian. Induction heating can be accomplished using a magnetic force, by means of a device external to the patient. The heating device may be caused to heat the implant device and thus heat (and treat) the tissue creating localized necrosis, and then be easily removed from the vicinity of the patient to stop the heating. In advanced versions of this implementation, the heating device and the implant device may be tuned such that only one implant device is heated at a time, if multiple implants have been deployed.

FIGS. 9-11 provide different embodiments of an implant device, according to aspects of the present invention.

Regarding the embodiment of FIG. 9, in this implant device 900, there are two c-rings 910, 920. C-ring 910 is one end of implant device 900 and C-ring 920 is the other end of the implant device 900. C-ring 910 has two overlapping ends 911, 912 and c-ring 920 also has two overlapping ends 921, 922. In this embodiment, c-rings 910 and 920 have diameter of about 20 mm, and each has a total length of about 2 cm. While ends A, 13 are shown to be the same in FIG. 9, in other embodiments, ends A, 13 can have different diameters and different lengths.

C-rings 910, 920 are coupled together by a spine 930, comprising a first curved member 932 and a second curved member 934. The two curved members 932, 934 are spaced apart (or bowed) at an intermediate area and come together at or near the two c-rings 910, 920.

FIGS. 10A through 10D show four different views of another implant device embodiment. In this embodiment, the implant device 1000 includes two c-rings 1010, 1020, coupled together by a spine 1030, comprising a first curved member 1032 and a second curved member 1034. Between the first and second curved members 1032, 1034, the c-rings 1010, 1020 overlap, with ends 1011, 1021 and ends 1021, 1022, respectively.

FIG. 10A is a top/side view; FIG. 10B is a perspective view; FIG. 10C is a front view; and FIG. 10D is another side view. In this embodiment, the first and second curved members 1032, 1034 do not meet each other. The first and second curved members 1032, 1034 each connect each of c-rings 1010, 1020, but on opposite sides of the overlap of the ends c-rings 1010, 1020. In this embodiment, again the first and second curved members 1032, 1034 bow away from each other, but this need not be the case in other embodiments.

FIGS. 11A through 11D show four different views of another implant device embodiment. In this embodiment, the implant device 1100 includes two helical ribbon spines 1132, 1134, connected by two c-rings 1110, 1120. In this embodiment, the two helical ribbon spines 1132, 1134 do not meet each other. FIG. 11A is a top/side view; FIG. 11B is a perspective view; FIG. 11C is a front view; and FIG. 11D is another side view. Each spine 1132, 1134 helically wraps around a central axis X of the implant device 1100, about half way around in this embodiment.

In this embodiment, the two helical ribbon spines 1132, 1134 are counter rotated to each other and connect at opposite sides of each of the c-rings 1110, 1120, e.g., like a basket. But the helical ribbon spines 1132, 1134 are twisted in a large pitch helical and bow in the center. The idea is that radial compression of the bowed splines as placed into a tubular vessel would rotate the helical ends of the ribbon located on each end to open or radially dilate the ends of the implant locking it into place.

Within the vessel, in various embodiments, the spines in the middle of the implant device would work similar to an old Wallstent, i.e., squeeze the middle of the Wallstent and it elongates. With the proposed implant device design, squeeze the bowed spines in the middle of the implant and it radially dilates the ribbon helices on each end of the implant device.

FIG. 12 is a schematic view of an implant device, such as implant device 1000, deployed within a mammalian body, in accordance with aspects of the present invention. Here, the implant device 1000 is shown being deployed to treat Abdominal Aortic Aneurysms (AAA).

One or more implant device may be deployed in various ways. For example, a delivery system, such as a catheter system, can have distal and proximal ends, where the distal end employs an atraumatic distal tip and the proximal end includes a handle. The delivery system can further include a catheter shaft having a tubular or other geometric shape catheter structure traversing from the proximal end to the distal end. A guide wire lumen can include a luminal space to enable passage accommodating a range of guide wire sizes. In one embodiment, the guide wire lumen is furthermore capable of being advanced distally or proximally to enable deployment of the implant attached along the external surface of the guide wire lumen and contained within the inner surface of the outer catheter shaft. In some embodiments, the delivery system catheter may employ a flexible distal segment and a steering wire anchored at the distal portion of the delivery catheter.

In some implementations, the delivery system, or other similar device, may allow a degree of recapture to occur in order to fix incorrect implanted device placements within the mammalian body, or devices implanted for temporary uses. For example, where the implant device is pushed through a tube for delivery, the same tube may be used to deliver a small wire equipped with maneuverable jaws at its distal end that could grab and remove or reposition an implanted device. In some cases, for example, a modified guide wire may be employed. In the same way, a ratchet sleeve with incorporated balloon may provide this function as well.

Multiple implant devices may be delivered in a single surgical operation (see, e.g., FIGS. 6 and 8). For example, in such a procedure, magnetic resonance imaging (MRI) may be employed initially in order to determine sizes of the vessel. The above procedure of deployment may only require, e.g., 10 to 20 minutes.

FIG. 13 is an embodiment of a catheter that can be used as a delivery mechanism. The catheter includes a handle 1310 with a knob 1312 to control delivery of a distal end delivery portion 1318, here a basket or balloon catheter. Extending from the handle is an outer shaft 1314, within which is disposed a telescoping inner shaft 1316. The delivery portion 1318 is coupled to a distal end of the inner shaft 1316. At a distal end of the delivery portion 1318 can be a pigtail 1320. An implant device in accordance with the present invention, such as those described above, can be mounted on the basket catheter for delivery inside the mammalian body to the delivery site, e.g., sites like those shown in FIGS. 6-8 and 12.

For example, one or more of the c-rings may revolve around a central axis of a catheter 1, 1.5, 2, 3, or more times. In this way, even when placed in larger chambers, the available expansion of the barbs or tissue penetration elements may cause an effective pressure to affix the tissue and implant for final deployment and release into said appendage.

The implant device may extend through the distal portion of the catheter, maintained on delivery portion 1318, and may further extend a short distance from the distal end of the end of the catheter during deployment. The distal end of the delivery system may also include a design where the catheter distal end is in a straight or neutral position and then steered using knobs and/or levers on the handle to create the pigtail distal segment 1320. Another lever located on the handle may be employed to deflect or steer the distal segment for cannulation of each pulmonary vein. This design may also include a plurality of electrodes to enable intra-cardiac electrogram interpretation.

In the above implementation, a spiral or pig-tail end can be included that allows the implant device to be delivered in a controlled manner and which protects the endocardial surface of the vein. The distal end of the delivery system may be employed for diagnostic purposes, such as ECG mapping of the vessel prior to and after implanting the device using electrodes, for example. The distal end may also allow a user to recapture the implant using devices described below if it is partially or already deployed, enabling further control and proper placement within a vessel.

By pushing the implant device out of the distal end of the catheter, the same may take up a position within a vessel as desired. One purpose of the PeBax pigtail is to protect the vessel or chamber during deployment in the same way, e.g., a Lasso® catheter does. In addition, the PeBax pigtail may be equipped with electrodes to allow mapping and/or ablation. The pitch of the distal loop or pigtail may be altered in known manner, e.g., by a control wire, to allow different cardiac geometries to be accommodated. Where mapping electrodes are used, their length may range, e.g., from approximately 0.5-4.0 mm, as an example.

In various embodiments, a rectangular lumen may be employed to contain and deliver the implant and a circular or oval lumen may be employed to contain signal wires for the mapping and ablation electrodes. It will be understood that the shape of the lumens may vary. In this way, mapping may be accomplished prior to deployment of the implant into an appendage. In addition, it will be understood that more than one rectangular or circular lumens may be employed, and their shapes may differ, according to the needs of any given catheter design. In systems where the catheter is made fully steerable or deflectable, additional lumens may be employed to provide the necessary control wires for steering or deflection.

In various embodiments, a handle includes a knob that can be separated by a distance L. The distance L can be chosen to allow for complete deployment of the implant device. A layer of epoxy may seal the handle to the sheath. The sheath can terminate at a distal end at a distal end bushing. A hypo-tube stock sleeve can be provided that surrounds a layer of epoxy, which can be used to hold a NiTi tension band. The distal end bushing is coupled to the sheath by a layer of epoxy. A distal end of the NiTi tension band may terminate at a hypo-tube and can be held in place by a layer of epoxy. A heat shrink can be set around the assembly.

In some embodiments, the delivery system may employ a small device, i.e., a ratchet slip joint and extension, within the delivery catheter or sheath that can provide a ratcheting function. In this way, the handle may be simplified, and provided with greater control, by having the operator only have to provide a repeated short-stroke motion to controllably cause the implant to exit the sheath and become implanted in the body.

In various embodiments, a balloon or other expandable delivery portion may be inflated or expanded with the implant device crimped (or compressed, or rolled up) or mounted to the interior or exterior of said balloon for delivery into the body. The c-rings of the implant device may be compressed and attached to the delivery portion for delivery within a body and into an appendage, vessel, or chamber. By placing a tip of the implant device, e.g., the proximal tip, into the vessel then expanding said balloon or dilatation mechanism of the catheter to dilate said implant containing a ratchet element, and inflating the balloon to fill up the interstitial space and obtain apposition of said implant device with ratchet mechanism against appendage wall or tissue, the implant device may be effectively held between the balloon and the wall of the vessel or tissue.

In another embodiment, the inflation lumen and balloon may be used to inflate the balloon for delivery while the telescopic action of the delivery system allows the implant device to latch the ends, such as to form a small coil from a helical shaped implant device to compress the shape of appendage and seal the entry to said appendage.

In yet another embodiment, a small diagnostic mapping catheter may be delivered to the target vessel (delivery site) through the lumen of said delivery catheter. The small loop or circular mapping catheter may be used to map the vessel before, during, and after deployment of said implant device and before, during, or after said balloon is inflated for delivery of said implant device to occur.

In various embodiments, the implant device includes at least one spine and at least one c-ring with optional tissue penetration elements around a threaded mandrel and confined by an outer tube and may be heat treated, or many other methods maybe used to obtain the final deployment ready state of said implant device or to obtain a desired memory shape. Removal of the outer tube allows the delivered implant device to spring away from the mandrel by virtue of its shape-memory character to achieve a deployed state.

In various embodiments, a deployment or delivery method may comprise a sequence of deployment steps. In general, removing the outer tube causes immediate deployment, resulting in impingement of the implant device against a vessel wall and an increase in the volume of said vessel or chamber by the implant device. In another embodiment, also illustrating a sequence of deployment steps, in this case which deploys the implant perpendicularly to the direction of implantation. This deployment direction may be useful in certain patient anatomies. In various embodiments, the implant device can emerge directly (and initially linearly) out of the distal tip of the catheter. In other embodiments, it will be understood that the order may be reversed.

In various embodiments, the implant device may be deployed from the proximal side first, such as at the ostium of the pulmonary vein/atrial junction, followed by deployment of an additional implant device within the vessel, appendage or other internal body part. The reason this may be advantageous is that this can allow more mechanical force to be applied to the luminal surface of the vessel tissue. This unwinding deployment allows installation of an implant that can provide sufficient mechanical force to achieve the clinical response necessary, without damaging the surrounding tissue. In addition, during deployment, e.g., while the implant device is partially deployed, the action of the partial implant on the electrical signal propagation may be confirmed or verified.

To deploy the distal end first, a split catheter shaft may be employed, such that separation of the catheter shaft at a location near the distal end causes the distal end to be deployed first. Of course, in certain implementations, the proximal end may also be deployed first. Such a split catheter shaft may be employed in various delivery system and methods described herein, or otherwise understood to be within the scope of the present invention. In this implementation, the distal end of the catheter may employ a polymer tip and guide wire for atraumatic delivery, and the polymer tip may be radiopaque. As with various embodiments described, the catheter may be delivered over a guide wire.

While the foregoing has described what are considered to be the best mode and/or other preferred embodiments, it is understood that various modifications can be made therein and that the invention or inventions may be implemented in various forms and embodiments, and that they may be applied in numerous applications, only some of which have been described herein. It is intended by the following claims to claim that which is literally described and all equivalents thereto, including all modifications and variations that fall within the scope of each claim.

1. An implant device, comprising:
at least one spine; and
at least two c-rings coupled together by the at least one spine.

2. The implant device of claim 1, wherein the at least two c-rings are maintained in a substantially parallel orientation by the at least one spine.

3. The implant device of claim 1, wherein ends of at least one c-ring overlap in a slip ring closure.

4. The implant device of claim 1, wherein ends of at least one c-ring have a cut ring closure, wherein the ends do not overlap.

5. The implant device of claim 1, wherein ends of at least one c-ring have mating teeth.

6. The implant device of claim 1, wherein one or more of the at least two c-rings are configured to self-adjust to accommodate a shape of a target location within a vessel of a body.

7. The implant device of claim 1, wherein one or more of the c-rings has a memory shape and can take a compressed state when constrained and automatically expands when such constraint is removed.

8. The implant device of claim 1, further comprising:
at least one stabilizer element coupled to at least one c-ring.

9. The implant device of claim 8, wherein the stabilizer element is a wire laterally extending from the at least one c-ring.

10. The implant device of claim 8, wherein the stabilizer element is a wire formed in the shape of a zigzag.

11. The implant device of claim 1, further comprising at least one tissue penetration element configured to increase frictional or mechanical resistance against inner walls of a vessel.

12. The implant device of claim 1, wherein the at least two c-rings includes an end c-ring having a funnel-like shape.

13. The implant device of claim 12, wherein the funnel-like shape is configured to conform to an ostium or antrum of a vessel.

14. The implant device of claim 1, wherein the at least two c-rings have fingers disposed around a common central axis.

15. The implant device of claim 14, wherein the at least one spine includes at least two helical ribbon spines that wrap at least partially around the common central axis.

16. The implant device of claim 15, wherein the at least two helical ribbon spines bow out and away from the common central axis.

17. The implant device of claim 1, wherein the at least one spine includes a plurality of spines that couple together the at least two c-rings.

18. The implant device of claim 17, wherein two or more of the plurality of spines couple together two c-rings.

19. The implant device of claim 1, further comprising a biologically inert coating.

20. The implant device of claim 1, further comprising a coating including one or more of drugs, biologics, chemicals, or combinations of one or more thereof.

21. The implant device of claim 1, further comprising a coating including a chemical ablation reagent.

22. The implant device of claim 1, wherein the at least one spine and the at least two c-rings are formed from a single sheet of material.

23. The implant device of claim 1, wherein the implant device is configured to deliver a force against tissue of the vessel in a range of between about 0.5 g/mm² and about 300 g/mm².

24. The implant device of claim 1, wherein the implant device is configured to deliver a radial force against tissue of a mammalian vessel at a target location that is sufficient to cause necrosis or apoptosis in the tissue in a deployed state, the necrosis or apoptosis sufficient to or delay electrical, neurological signal conduction traveling along an axis of the vessel and/or within an adjacent chamber in mammalian.

25. The implant device of claim 1, wherein the implant device is configured to deliver a radial force against tissue of a mammalian vessel at a target location that is sufficient to compress at least one Ion channel in the adjacent tissue sufficient to delay electrical or neurological signals traveling along an axis of the vessel and/or within an adjacent chamber in mammalian.

26. The implant device of claim 1, further comprising:
a micro-circuit configured to measure or monitor a value of electrical conduction propagating along the axis of a mammalian vessel within which the implant device is deployed.

27. The implant device of claim 26, further comprising:
one or more electrodes disposed in or on the at least one substrate and in communication with the micro-circuit, the one or more electrodes configured for sensing conditions within the vessel and/or delivering energy to the vessel.

28. The implant device of claim 27, wherein the one or more electrodes include ablation electrodes, mapping electrodes, or ablation and mapping electrodes.

29. The implant device of claim 26, wherein the micro-circuit is configured to use an Ionic exchange with the vessel to charge a battery of the micro-circuit.

30. The implant device of claim 26, wherein the micro-circuit is configured to measure and/or monitor a value of electrical conduction propagating along the axis of the vessel.

31. The implant device of claim 30, wherein the micro-circuit is further configured to wirelessly transmit an indication of the electrical conduction in mammalian.

32. The implant device of claim 26, wherein the micro-circuit is configured to receive an electromagnetic signal and to inductively heat the vessel in response to the electromagnetic signal.

33. A method of making an implant device, comprising:
providing at least one spine;
providing at least two c-rings; and
using the spine to couple together the at least two c-rings.

34. The method of claim 33, further comprising maintaining the at least two c-rings in a substantially parallel orientation using the at least one spine.

35. The method of claim 33, further comprising forming ends of at least one c-ring overlap in a slip ring closure.

36. The method of claim 33, further comprising forming ends of at least one c-ring have a cut ring closure, wherein the ends do not overlap.

37. The method of claim 33, further comprising forming ends of at least one c-ring have mating teeth.

38. The method of claim 33, further comprising forming one or more of the at least two c-rings to be self-adjusting to accommodate a shape of the target location within the vessel of a body.

39. The method of claim 33, further comprising processing the one or more of the c-rings to have a memory shape that automatically expands when transitioned from a compressed state to a deployed state.

40. The method of claim 33, further comprising providing at least one stabilizer element extending from at least one c-ring.

41. The method of claim 40, further comprising forming the stabilizer element from a wire laterally extending from the at least one c-ring.

42. The method of claim 40, further comprising forming the stabilizer element in the shape of a zigzag.

43. The method of claim 33, further comprising providing at least one tissue penetration element configured to increase a frictional or mechanical resistance against inner walls of the vessel.

44. The method of claim 33, further comprising forming an end c-ring from the at least two c-rings to have a funnel-like shape.

45. The method of claim 44, further comprising forming the funnel-like shape to conform to an ostium or antrum of the vessel.

46. The method of claim 33, further comprising forming the at least two c-rings to have fingers disposed around a common central axis.

47. The method of claim 46, further comprising forming the at least one spine to include at least two helical ribbon spines that wrap at least partially around the common central axis.

48. The method of claim 47, further comprising forming the at least two helical ribbon spines bow out and away from the common central axis.

49. The method of claim 33, wherein the at least one spine includes a plurality of spines that couple together the at least two c-rings.

50. The method of claim 49, further comprising using two or more of the plurality of spines to couple together two c-rings.

51. The method of claim 33, further comprising providing a biologically inert coating to the implant device.

52. The method of claim 33, further comprising providing a coating to the implant device including one or more of drugs, biologics, chemicals, or combinations of one or more thereof.

53. The method of claim 33, further comprising providing a chemical ablation reagent coating to the implant device.

54. The method of claim 33, further comprising forming the at least one spine and the at least two c-rings from a single sheet of material and then shaping the material into the implant device form.

55. The method of claim 33, further comprising forming the implant device to deliver a force against tissue of the vessel in a range of between about 0.5 g/mm² and about 300 g/mm².

56. The method of claim 33, further comprising:
providing a micro-circuit in or on the at least one spine and/or at least one of the at least two c-rings that is configured to measure or monitor a value of electrical conduction propagating along the axis of a mammalian vessel within which the implant device is deployed.

57. The method of claim 56, further comprising:
disposing one or more electrodes in or on the at least one spine and/or at least one of the at least two c-rings that are in communication with the micro-circuit, the one or more electrodes configured for sensing conditions within the vessel and/or delivering energy to the vessel.

58. The method of claim 57, wherein the one or more electrodes include ablation electrodes, mapping electrodes, or ablation and mapping electrodes.

59. The method of claim 56, wherein the micro-circuit is configured to use an Ionic exchange with the vessel to charge a battery of the micro-circuit.

60. The method of claim 56, wherein the micro-circuit is configured to measure and/or monitor a value of electrical conduction propagating along the axis of the vessel.

61. The implant device of claim 60, wherein the micro-circuit is further configured to wirelessly transmit an indication of the electrical conduction in mammalian.

62. The method of claim 56, wherein the micro-circuit is configured to receive an electromagnetic signal and to inductively heat the vessel in response to the electromagnetic signal.

63-95. (canceled)
